(19) United States
(12) Patent Application Publication
(10) Pub. No.: US 2024/0066043 A1
(43) Pub. Date: Feb. 29, 2024

(54) PHARMACEUTICAL COMPOSITIONS CONTAINING PSILOCYBIN FOR IMPROVED DISSOLUTION AND RAPID ONSET BY TARGETED STOMACH AND MUCOSAL RELEASE

(71) Applicant: Xpira Pharmaceuticals Inc., Toronto (CA)

(72) Inventors: Maria Vanesa Fernandez CID, Haarlem (NL); Nicholas Jo Lic WONG, North York (CA)

(73) Assignee: Xpira Pharmaceuticals Inc., Toronto (CA)

(21) Appl. No.: 18/447,553

(22) Filed: Aug. 10, 2023

Related U.S. Application Data
(60) Provisional application No. 63/371,408, filed on Aug. 15, 2022.

(51) Int. Cl. A61K 31/675; A61K 9/00; A61K 47/32; A61K 47/36

(52) U.S. Cl. CPC A61K 31/675; A61K 9/0053; A61K 47/32; A61K 47/36

(57) ABSTRACT

Pharmaceutical compositions are provided containing psilocybin, which are designed for rapid dissolution properties and immediate or rapid release. The compositions are designed for targeted release by way of: (1) stomach release; or (2) mucosal release. The compositions can be used as pre-formulations for further tableting, encapsulation, or formulating into a sublingual dosage form for oral use, designed for rapid therapeutic onset. Particularly, the compositions overcome unfavourable formulation characteristics of psilocybin that cause drug release variability and problems for formulating into finished dosage forms. More particularly, psilocybin is formulated in a manner to overcome drug release variability thereby providing uniform potency and desirable bioavailability characteristics. The compositions may be used for inducing therapeutically beneficial behaviour change in subjects with a variety of psychiatric conditions.

### CROSS REFERENCE TO RELATED APPLICATIONS

This application claims priority from U.S. Patent Application No. 63/371,408, filed on Aug. 15, 2022, the entirety of which is hereby incorporated by reference.

### FIELD OF INVENTION

The present invention relates to pharmaceutical compositions comprising psilocybin, which are designed for rapid dissolution properties and rapid release, and in particular, targeted stomach release or mucosal release.

### BACKGROUND

### Psilocybin and Its Properties

Psilocybin, 3[2-(dimethylamino)ethyl]-1H-indol-4-yl] dihydrogen phosphate, is a natural product produced by numerous species of Psilocybe mushrooms. It is a tryptamine derivative, and in humans the phosphate group is rapidly enzymatically cleaved in the body to produce psilocin, an agonist at a variety of serotonin receptors, the most important of which in this setting is the 5-HT2A receptor (Carhart-Harris et al., 2014; Nichols, 2004).

Psilocybin has the following structure:

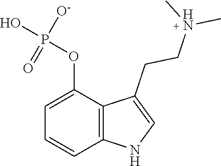

Psilocybin was first isolated from Psilocybe mushrooms in 1957, followed by de novo synthesis in 1958 (Passie, Seifert, Schneider, & Emrich, 2002). It was marketed worldwide in the 1960s as Indocybin™ for experimental and psychotherapeutic purposes. Although it was well tolerated and demonstrated potentially useful effects, it was classified as a controlled substance in the U.S., placed in Schedule I in 1970, and effectively removed from clinical use or scientific study.

Psilocybin is a tryptamine derivative presenting as a white crystalline solid with a melting point of 220-228° C. It is stable over extended periods in dark storage at controlled room temperature. Psilocybin is soluble in 20 parts boiling water or 120 parts boiling methanol.

Oral psilocybin has about a 50% bioavailability and psilocin is detectable in plasma within 20 minutes of administration of the parent compound (Brown, et al., 2017; Hasler, Bourquin, Brenneisen, Bar, & Vollenweider, 1997). The half-life of psilocin in blood is 2-3 hours. Onset of noticeable psychoactive effects occurs within one hour, peaks at about two hours after a dose, and loss occurs typically around six hours after the dose.

Based on this time course, protocols mandate observation in the clinical trial setting until approximately 8 hours after dosing. Further, exposure following a 25 mg oral dose is associated with both near-maximal occupancy of neocortical serotonin 5-HT2A receptors and subjective intensity ratings of elements of the psychedelic experience that have been repeatedly associated with longer-term therapeutic benefits (Madsen et al., 2019). As a result, it is desirable to have a composition of psilocybin with a precise release profile for contemporaneous effect with the timing of the clinical observation.

Psilocybin reliably induces profound changes in sensory perception, emotion, thought, and sense of self, characterized by marked alterations in all mental functions, including perception, mood, volition, cognition, and self-experience (Geyer & Vollenweider, 2008; Studerus, Kometer, Hasler, & Vollenweider, 2011). These profound changes are often referred to as mystical-type experiences.

Measures of mystical-type experience occurring during psilocybin treatment have been repeatedly observed to predict later effects on behaviour and emotions, including reductions in depressive and anxious symptoms (Griffiths et al., 2016; MacLean, Johnson, & Griffiths, 2011; Ross et al., 2016). Non-clinical in vivo and in vitro studies, found via literature searches, demonstrate that, similar to humans, when psilocybin is administered orally to rats it is rapidly dephosphorylated to psilocin in the intestinal mucosa by alkaline phosphatase and a nonspecific esterase, with approximately 50% of the total volume of psilocin absorbed from the digestive tract (Kalberer et al., 1962).

Maximum plasma levels are achieved after approximately 90 minutes (Chen et al., 2011). When administered systemically (i.e., bypassing the gut), initial psilocybin metabolism is performed by tissue phosphatases, with in vitro studies indicating the kidneys as being among the most active metabolic organs (Horita & Weber, 1961). Across species tested, the highest levels of psilocin were found in the neocortex, hippocampus, and thalamus (Hopf & Eckert, 1969).

Recent clinical studies utilizing pharmaceutical-grade oral psilocybin under controlled conditions have been performed upon healthy participants and various subpopulations to characterize the safety profile and evaluate clinical efficacy. Though the safety reporting criteria and the level of data verification varied greatly between studies, including many participant-reported outcomes, these data have been utilized to elucidate the expected adverse event profile of psilocybin.

Several lines of evidence suggest that serotonergic hallucinogens, such as psilocybin, have clinical potential for inducing therapeutically beneficial behaviour change in subjects with a variety of psychiatric conditions.

### Pharmacokinetics and Metabolism

Following oral administration of 0.224 mg/kg psilocybin (10 to 20 mg), average blood concentration of the active metabolite psilocin was calculated to be 8.2±2.8 ng/mL after 105±37 minutes, yielding an estimated dose-normalized bioavailability of psilocybin of 52.7±20% (N=3) (Hasler, Bourquin, Brenneisen, Bar, & Vollenweider, 1997).

Psilocin typically appears in plasma within 15 minutes after oral administration. Psilocin half-life following oral administration of psilocybin was found to be approximately 3±1.1 hours and is detectable for up to 24 hours after administration (Brown et al., 2017). The levels of psilocin peaked at approximately 80 minutes, but the peak psilocin concentration was more gradually attained in some subjects than in others, suggesting metabolism rates can vary between individuals (Brown et al., 2017). Plasma psilocin concentrations are directly correlated with neocortical 5-HT2A occupancy and subjective assessment of its psychoactive effects (Madsen et al., 2019).

Psilocin is metabolized to 4-hydroxyindole-3-acetic acid by deamination and demethylation via liver enzymes such as monoamine oxidase, and aldehyde dehydrogenase (FIG. 5.1-1, Hasler, Bourquin, Brenneisen, Bar, et al., 1997). Psilocin is also extensively glucuronidated by the UDP-glucuronosyltransferase (UGT) family of enzymes, with the highest glucuronidation activity demonstrated by UGT1A10 (Manevski et al., 2010). The amount of psilocin glucuronide excreted renally has been shown to exceed that of psilocin over a 24-hr time period, and analysis of psilocin in urine over 24 hours after a single dose has shown that less than 4% of the overall clearance of psilocin occurs through renal excretion (Hasler, Bourquin, Brenneisen, & Vollenweider, 2002). The pharmacokinetics of psilocybin (as psilocin) are linear over the dose range of 0.3-0.6 mg/kg (Brown et al., 2017; Hasler et al., 2002).

### SUMMARY OF THE INVENTION

Disclosed are pharmaceutical compositions containing psilocybin, preferably for use as pre-formulations for manufacturing dosage forms for oral administration (such as tablets and capsules), and more particularly, sublingual administration. The pharmaceutical compositions comprise psilocybin and one or more pharmaceutically acceptable excipients. In some embodiments, the pharmaceutical compositions can be prepared by carbon dioxide spray drying the psilocybin and the one or more pharmaceutically acceptable excipients to provide pharmaceutical compositions in particulate form.

In one aspect, disclosed is a pharmaceutical composition comprising, (a) psilocybin in an amount of about 5% w/w to about 15% w/w of the pharmaceutical composition; and (b) chitosan in an amount of about 85% w/w to about 95% w/w of the pharmaceutical composition; wherein the pharmaceutical composition has a dissolution performance such that about 80% or greater of the psilocybin in the pharmaceutical composition is released in a subject's stomach within the first 5 minutes of oral administration.

In an embodiment, the pharmaceutical composition may comprise (a) psilocybin in an amount of about 9.4% w/w to about 10.9% w/w of the pharmaceutical composition; and (b) chitosan in an amount of about 89.1% w/w to about 90.6% w/w of the pharmaceutical composition.

In an embodiment, the pharmaceutical composition has a dissolution performance such that about 85% or greater of the psilocybin in said pharmaceutical composition is released in a subject's stomach within the first 5 minutes of oral administration.

In an embodiment, the pharmaceutical composition is in particulate form, and wherein at least 90% (D90) of the pharmaceutical composition has a particle size of less than 3.37 μm. In a further embodiment, at least 90% (D90) of the pharmaceutical composition has a particle size between 1 to 3 μm. In a still further embodiment, at least 90% (D90) of the pharmaceutical composition has a particle size between 0.7-1.5 μm.

In an embodiment, the pharmaceutical composition further comprises one or more pharmaceutically acceptable excipients. In a further embodiment, the one or more pharmaceutically acceptable excipients are selected from the group consisting of: binders, disintegrants, lubricants, flowing agents, glidants, anti-adherents, surfactants, stabilizers, binders, bulking agents, colouring agents, and coating agents.

In an embodiment, the pharmaceutical composition is in a unit dosage form that is an oral dosage form. In a further embodiment, the oral dosage form may contain about of about 25 to about 30 mg psilocybin. In a still further embodiment, the oral dosage form may be a tablet or a capsule.

In a further aspect, disclosed is the use of the pharmaceutical composition comprising psilocybin and chitosan as described above, for the manufacture of an oral dosage form comprising the pharmaceutical composition and optionally, one or more pharmaceutically acceptable excipients, wherein the pharmaceutical composition has a dissolution performance such that about 80% or greater of the psilocybin in the oral dosage form is released in a subject's stomach within the first 5 minutes of oral administration.

In an embodiment, the pharmaceutical composition used in the manufacture of the oral dosage form comprises about 10% by weight psilocybin, about 90% of chitosan.

In an embodiment, the pharmaceutical composition used in the manufacture of the oral dosage form has a dissolution performance such that about 85% or greater of the psilocybin in the oral dosage form is released in a subject's stomach within the first 5 minutes of oral administration.

In an embodiment, the pharmaceutical composition used in the manufacture of the oral dosage form is in particulate form, and wherein at least 90% (D90) the pharmaceutical composition containing psilocybin have a particle size of less than 3.37 μm. In a further embodiment, at least 90% (D90) of the pharmaceutical composition has a particle size between 1 to 3 μm. In a still further embodiment, at least 90% (D90) of the pharmaceutical composition has a particle size between 0.7-1.5 μm.

In an embodiment, the oral dosage form is a tablet or capsule. In a further embodiment, the oral dosage form contains about 25 to about 30 mg of the psilocybin.

In another aspect, disclosed is a pharmaceutical composition comprising, (a) psilocybin in an amount of about 5 to about 15% w/w of the pharmaceutical composition; and (b) polyvinylpyrrolidone in an amount of about 85 to about 95% w/w of the pharmaceutical composition; wherein the pharmaceutical composition has a dissolution performance such that about 100% of the psilocybin in the pharmaceutical composition is released in a subject's oral mucosal wall within the first 1 minute of oral administration.

In an embodiment, the pharmaceutical composition comprises (a) psilocybin in an amount of 9.1% w/w of the pharmaceutical composition; and (b) polyvinylpyrrolidone in an amount of 90.9% w/w of the pharmaceutical composition.

In an embodiment, the pharmaceutical composition has a dissolution performance such that about 60% to about 85% of the psilocybin in the pharmaceutical composition is released in a subject's oral mucosal wall within the first 30 seconds of oral administration. In a further embodiment, the pharmaceutical composition has a dissolution performance such that about 85% or greater of the psilocybin in the pharmaceutical composition is released in a subject's oral mucosal wall within the first 30 seconds of oral administration.

In an embodiment, the pharmaceutical composition is in particulate form, and wherein at least 90% (D90) of the pharmaceutical composition has a particle size of less than 1.54 μm. In a further embodiment, at least 90% (D90) of the pharmaceutical composition has a particle size between 0.7-1.5 μm.

In an embodiment, the pharmaceutical composition further comprises one or more pharmaceutically acceptable excipients. In a further embodiment, the one or more pharmaceutically acceptable excipients are selected from the group consisting of: binders, disintegrants, lubricants, flowing agents, glidants, anti-adherents, surfactants, stabilizers, binders, bulking agents, colouring agents, and coating agents.

In an embodiment, the pharmaceutical composition is in a unit dosage form that is a sublingual dosage form. In a further embodiment, the sublingual dosage form contains about 25 to about 30 mg of the psilocybin. In a further embodiment, the sublingual dosage form is a tablet, a wafer, or a film.

In another aspect, disclosed is the use of the pharmaceutical composition comprising psilocybin and polyvinylpyrrolidone, as described above, for the manufacture of a sublingual dosage form comprising the pharmaceutical composition and optionally, one or more pharmaceutically acceptable excipients, wherein the pharmaceutical composition has a dissolution performance such that about 100% of the psilocybin in the pharmaceutical composition is released in a subject's oral mucosal wall within the first 1 minute of oral administration.

In an embodiment, the pharmaceutical composition used in the manufacture of the sublingual dosage form comprises about 10% by weight psilocybin and about 90% of polyvinylpyrrolidone.

In an embodiment the pharmaceutical composition used in the manufacture of the sublingual dosage form has a dissolution performance such that about 60% to about 85% of the psilocybin in the pharmaceutical composition is released in a subject's oral mucosal wall within the first 30 seconds of oral administration. In a further embodiment the pharmaceutical composition used in the manufacture of the sublingual dosage form has a dissolution performance such that about 85% or greater of the psilocybin in the pharmaceutical composition is released in a subject's oral mucosal wall within the first 30 seconds of oral administration.

In an embodiment, the pharmaceutical composition used in the manufacture of the sublingual dosage form is in particulate form, and wherein at least 90% (D90) the pharmaceutical composition containing psilocybin have a particle size of less than 1.54 μm. In a further embodiment, at least 90% (D90) the pharmaceutical composition containing psilocybin have a particle size between 0.7-1.5 μm.

In an embodiment, the sublingual dosage form contains about 25 to about 30 mg of the psilocybin.

In a further aspect, disclosed is a pharmaceutical composition for mucosal release, comprising psilocybin in an amount of about 9.1% w/w of the pharmaceutical composition; and polyvinylpyrrolidone; wherein the ratio psilocybin to polyvinylpyrrolidone is in a range of 1:10 to 1:11 of the pharmaceutical composition.

In an embodiment, the pharmaceutical composition further comprises one or more pharmaceutically acceptable excipients.

In a further aspect, disclosed is a pharmaceutical composition for stomach release, comprising psilocybin in an amount of about 9.6% w/w of the pharmaceutical composition; and chitosan; wherein the ratio of psilocybin to chitosan is in a range of 1:10 to 1:11 of the pharmaceutical composition. In an embodiment, the pharmaceutical composition further comprises one or more pharmaceutically acceptable excipients.

In a further aspect, disclosed is a pharmaceutical composition of particulate form comprising psilocybin, wherein at least 90% (D90) of the pharmaceutical composition containing psilocybin have a particle size of less than 3.37 μm.

In an embodiment, at least 90% (D90) of the pharmaceutical composition containing psilocybin have a particle size between 1.5-3 μm. In a further embodiment, at least 90% (D90) of the pharmaceutical composition containing psilocybin have a particle size of 0.7-1.5 μm.

In a further aspect, disclosed is the use of the pharmaceutical composition of particulate form comprising psilocybin, wherein at least 90% (D90) of the pharmaceutical composition containing psilocybin have a particle size of less than 3.37 μm, for the manufacture of a dosage form for oral administration. In a further embodiment, in addition to the pharmaceutical composition of particulate form comprising psilocybin, the dosage form for oral administration further comprises one or more pharmaceutically acceptable excipients.

In a further aspect, disclosed are oral dosage forms, such as tablets, capsules, or sublingual dosage forms or formulations prepared from any of the pharmaceutical compositions described above. In a further embodiment, the oral dosage form or the sublingual dosage form contain about 25 mg to about 30 mg of psilocybin.

### BRIEF DESCRIPTION OF THE FIGURES

FIG. 1 shows a photograph of (a) unformulated psilocybin (two views), (b) a chitosan-psilocybin sample prepared in accordance with the manufacturing processes disclosed herein, and (c) a polyvinylpyrrolidone (PVP)-psilocybin sample prepared in accordance with the manufacturing processes disclosed herein;

FIG. 2 is a line graph illustrating the release profile of the chitosan-psilocybin sample;

FIG. 3 is a line graph illustrating the release profile of the PVP-psilocybin sample;

FIG. 4A is a Scanning Electron Microscope (SEM) micrograph of unformulated psilocybin;

FIG. 4B is the SEM micrograph of FIG. 4A, annotated to include a scale indicating the crystal size. Psilocybin has a columnar and square like crystalline structure. The crystal size is approximately 30 μm or less;

FIG. 5 is a bar graph illustrating the particle size distribution of the chitosan-psilocybin sample; and

FIG. 6 is a bar graph illustrating the particle size distribution of the PVP-psilocybin sample.

### DETAILED DESCRIPTION

### Definitions

The term “psilocybin”, as used herein, refers to the compound having the following chemical structure.

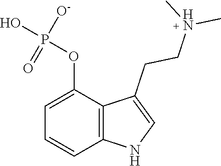

As used herein, “unformulated psilocybin” (also referenced as “neat psilocybin”) refers to psilocybin, which has not been combined with any other chemical components. Conversely, “formulated psilocybin”, as used herein, refers to a composition comprising psilocybin, wherein the psilocybin has been combined with one or more pharmaceutically acceptable excipients. The formulated psilocybin may be prepared, for example, by forming a solution of the psilocybin and one or more pharmaceutically acceptable excipients. The solution containing the psilocybin and one or more pharmaceutically acceptable excipients may be subjected to a spray drying process, such as supercritical carbon dioxide spray drying process to provide a powder form of the formulated psilocybin. In particular, the formulated psilocybin may be prepared using the manufacturing processes disclosed herein.

The term “acceptable” or “pharmaceutically acceptable”, with respect to a formulation, composition, or ingredient, as used herein, means having no persistent detrimental effect on the general heath of a subject being treated or does not abrogate the biological activity or properties of the compound, and its relatively non-toxic.

An “excipient”, as used herein, refers to a pharmaceutically acceptable ingredient that is commonly used in the pharmaceutical field for preparing granule and/or solid oral dosage formulations. Examples of categories of excipients include, but are not limited to, binders, disintegrants, lubricants, flowing agents, glidants, anti-adherents, surfactants, stabilizers, binders, bulking agents (also known as fillers, diluents, and carriers), colouring agents, and coating agents.

As used herein, a “pharmaceutical composition” refers to a mixture of a

pharmaceutically active ingredient (also referenced herein as active pharmaceutical ingredient) and one or more pharmaceutically acceptable excipients. The pharmaceutical compositions disclosed herein comprises psilocybin as the pharmaceutically active ingredient. The pharmaceutical compositions may be prepared by formulating the psilocybin with the one or more one or more pharmaceutically acceptable excipients, as discussed above for “formulated psilocybin”.

The pharmaceutical composition may be used as a pre-formulation. As used herein, a “pre-formulation” means a composition that can be further processed, (e.g., tableting, encapsulation) or further formulated with one or more further pharmaceutically acceptable excipients to yield a dosage form intended for administration or consumption.

The term “unit dosage form” as used herein refers to physically discrete units suited as unitary dosages for a subject to be treated; each unit containing a predetermined quantity of a pharmaceutically active ingredient calculated to produce the desired therapeutic effect in association with the one or more pharmaceutically acceptable excipients. The choice of a dosage form will depend on the route of administration.

As used herein, the term “oral administration” means a route of administration, whereby by a substance is taken through the mouth. Oral administration may involve swallowing, so that the substance (such as a pharmaceutically active ingredient) enters the gastrointestinal tract, or buccal or sublingual administration by which the substance (such as a pharmaceutically active ingredient) enters the blood stream directly from the mouth. Examples of dosage forms for oral administration involving swallowing, include but are not limited to, tablets and capsules. Examples of dosage forms for sublingual administration, include but are not limited to, tablets, wafers, and films.

The term “rapid release” (also referenced herein as “immediate release”) refers to the release of a majority of the pharmaceutically active ingredient, e.g. greater than about 50%, about 55%, about 60%, about 65%, about 70%, about 75%, about 80%, or about 90% within a relatively short time, e.g. within 1 hour, 40 minutes, 30 minutes, 20 minutes, 10 minutes, 5 minutes or about 1 minute after the pharmaceutically active ingredient has been administered orally, or more particularly, sublingually.

Rapid release pharmaceutical compositions comprising psilocybin and one or more pharmaceutically acceptable excipients may be formulated particularly for stomach release. As used herein “stomach release” means that a majority of the psilocybin, following oral administration the pharmaceutical composition, is absorbed in a subject's gastrointestinal tract. The rapid release pharmaceutical compositions disclosed herein, comprising psilocybin and one or more pharmaceutically acceptable excipients suitable for stomach release, have a dissolution performance such that about 80% or greater of the psilocybin in the pharmaceutical composition is released in a subject's stomach within the first 5 minutes of oral administration.

Rapid release pharmaceutical compositions comprising psilocybin and one or more pharmaceutically acceptable excipients may also be formulated for mucosal release. As used herein, “mucosal release” means that a majority of the psilocybin following administration (such as by sublingual administration) of the pharmaceutical composition is absorbed by a subject's oral mucosa for systemic delivery. The rapid release pharmaceutical compositions disclosed herein, comprising psilocybin and one or more pharmaceutically acceptable excipients suitable for mucosal release, have a dissolution performance such that about 100% of the psilocybin in the pharmaceutical composition is released in a subject's oral mucosal wall within the first 1 minute of oral administration, and preferably about 85% or greater of the psilocybin in the pharmaceutical composition is released in a subject's oral mucosal wall within the first 30 seconds of oral administration.

As used herein, the term “subject” (also referenced herein as a “patient”) refers to an animal which is the object of treatment, observation or experiment. For example, a subject may be, but is not limited to a mammal, including a human.

The terms “treat”, “treating” or “treatment”, as used herein, include alleviating, abating or ameliorating a disease or condition symptoms, preventing additional symptoms, ameliorating or preventing the underlying causes of symptoms, inhibiting the disease or condition, e.g. arresting the development of the disease or condition, relieving the disease or condition, causing regression of the disease or condition, relieving a condition caused by the disease or condition, or stopping the symptoms of the disease or condition. The terms “treat”, “treating” or “treatment”, include, but are not limited to, prophylactic and/or therapeutic treatments.

### Problematic Composition Qualities

Due to the psychoactive nature of psilocybin and vulnerabilities of the patient to be treated, including the emotional/cognitive/behavioural state/mindset of the patient, the safety of participants in clinical trials is paramount; there exists a need for a dosage form of psilocybin providing reproducible and precise delivery profile.

Certain physical properties of psilocybin present a challenge in developing compositions suitable for preparing an oral dosage form (e.g., tablet or capsule) having both a suitable quantity of active agent and a small enough tablet mass to allow ease of swallowing.

Psilocybin is, for example, a fluffy material, a very fine powder crystal, with relatively low bulk density and with very poor flow properties, which tends to agglomerate. These properties make it difficult to formulate the drug into a small oral dosage form with uniformity of weight, hardness, and other desirable tablet properties. Further, these properties of unformulated psilocybin make it difficult to handle and hard to add in the preparation of a solution prior to spray drying for manufacturing a finished dosage form.

In this regard, Table 1 sets out the measured bulk density, tap density, Hausner ratio and compressibility index of unformulated psilocybin versus formulated psilocybin prepared by the process as detailed below. Unformulated psilocybin has the highest Hausner ratio, representative of the poorest flowability compared to formulated psilocybin.

TABLE 1
Flowability Measurements

| | Bulk | Tapped | | Compressi- |
|---|---|---|---|---|
| | density | density | Hausner | bility |
| Compound | [g/ml] | [g/ml] | ratio | index |
| Chitosan- | 0.33 | 0.48 | 1.48 | 32% |
| Psilocybin | | | | |
| PVP-Psilocybin | 0.22 | 0.36 | 1.65 | 39% |
| Psilocybin | 0.19 | 0.45 | 2.36 | 58% |

The Hausner ratio reported in Table 1 above is a number that is correlated to the flowability of a powder or granular material. The Hausner ratio is expressed as the tap density divided by the bulk density, Hr=ρtap/ρb, and related Carr index,? CI=1−1/Hr, are used to indicate the flowability of granular powders in a wide variety of industries. The measurements of the Hausner ratio above were calculated in accordance with USP41-NF36, Powder flow 1174 Compressibility index and Hausner ratio, p. 7483. The densities of the powers were calculated in accordance with USP41-NF36, Physical tests 616, Bulk density and tapped density of powders, p. 6360.

In addition, psilocybin has other undesirable flow characteristics. For example, psilocybin is sticky and can adhere to surfaces such as tablet punch faces and dies, causing problems in tableting, especially on a high-speed tablet press. These properties of psilocybin may also result in the adherence of the drug substance to the walls of capsules, resulting in uneven dissolution and uptake in the gastrointestinal tract.

The disclosed pharmaceutical compositions and manufacturing processes overcome these problems by preformulating the psilocybin drug substance prior to mixing with flowing agents or bulking agents for final composition, thereby avoiding uneven uptake of the drug when administered to a patient.

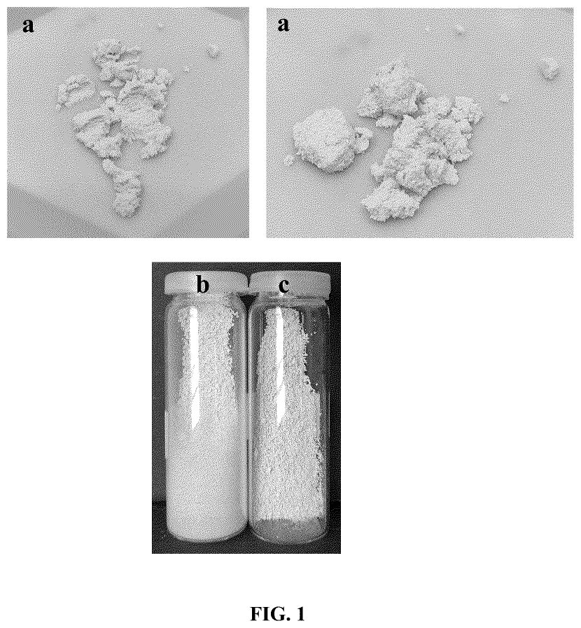

In this regard, FIG. 1 shows the neat, unformulated psilocybin and how it forms big lumps of powder on the sample tray and the fine and free flowing powders obtained after spray drying in accordance with the disclosed manufacturing processes. In an embodiment, the manufacturing process comprises 3 steps: solution making, spraying the solution with supercritical carbon dioxide to create powder and collection of the powder. The solution comprises the active ingredient, excipient and solvent, wherein the amount of solvent in the solution is determined based on the solubility of the excipient or active ingredient, as applicable, and the viscosity of the created solution. The solution is then sprayed through a nozzle (e.g., orifice size of 0.5-1.5 mm) to a pressure vessel (e.g., 10 L in volume, with a pressure of 150 bar) filled with supercritical carbon dioxide, which creates a free-flowing powder. At the end of the process, the powder is collected in a collection chamber.

Prepared in accordance with the disclosed manufacturing processes, the resulting pharmaceutical compositions containing psilocybin (1) have a minimal mass of added excipients, thereby allowing preparation of small, easily administered (e.g., by swallowing) dosage forms which enhance patient acceptance and compliance, and yet also (2) have excellent properties for formation of a tablet, capsule, or sublingual dosage form, and (3) provide tablets with excellent wetting, disintegration, and ultimately, rapid and excellent drug release properties.

### Pharmaceutical Compositions

Pharmaceutical compositions containing psilocybin are described in further detail as follows. The components employed in the pharmaceutical compositions as described herein should be pharmaceutically acceptable.

The pharmaceutical compositions can be used as pre-formulations for further tableting, encapsulation, or formulating into a sublingual dosage form for oral use, designed for rapid therapeutic onset. Particularly, the pharmaceutical compositions described herein overcome unfavourable formulation characteristics of psilocybin that cause drug release variability and problems for formulating into finished dosage forms. More particularly, psilocybin is formulated in a manner to overcome drug release variability thereby providing uniform potency and desirable bioavailability characteristics. The pharmaceutical compositions may be used for inducing therapeutically beneficial behaviour change in subjects with a variety of psychiatric conditions.

In preferred embodiments, the pharmaceutical compositions containing psilocybin are suitable for mucosal release of the psilocybin to be absorbed by oral mucosa or stomach release of the psilocybin to be absorbed in the gastrointestinal tract.

### Composition for Mucosal Release

The pharmaceutical compositions containing psilocybin can be suitable for mucosal release so that the psilocybin can be absorbed by oral mucosa for systemic delivery. The pharmaceutical compositions containing psilocybin can be suitable for sublingual administration. In one embodiment, the pharmaceutical compositions containing psilocybin utilize excipients (such as coatings) that are pharmaceutically acceptable and can be dissolved in water on pH 6.5-7.5, similar to the pH of the human mouth. Further, suitable excipients (such as coatings) should dissolve or substantially disintegrate in water or release the psilocybin quickly enough to achieve an immediate uptake in the mouth. For example, suitable excipients include polyvinylpyrrolidone (also known as PVP) and (hydroxypropyl)methyl cellulose, which are pH-independent, water-soluble materials. Another example of a suitable excipient is modified starch, which facilitates the release of an active pharmaceutical ingredient (API) in water by creating a colloidal system in the water. All three candidates have zero caloric value when included in the pharmaceutical composition in the conventional amount to be used in the mouth. Such pharmaceutical compositions can be used as pre-formulation compositions, which are used in the manufacture of pharmaceutical dosage forms.

In an embodiment, a pharmaceutical composition for mucosal release may comprise psilocybin and polyvinylpyrrolidone, wherein the ratio psilocybin to polyvinylpyrrolidone in a range of 1:10 to 1:11 of the pharmaceutical composition. In a further embodiment, the psilocybin is in an amount of about 9.1% w/w of the pharmaceutical composition.

Examples of pre-formulation compositions containing psilocybin as the API, which are suitable for mucosal release are provided in Table 3 below.

TABLE 3
Pre-formulation compositions for mucosal release

| | | Excip. conc. | Active conc. |
|---|---|---|---|
| Sample | Excipient | wt. % | Wt. % |
| 1 | Hydroxypropyl | 90.4% | 9.6% |
| | methylcellulose | | |
| | (“HPMC”) | | |
| 2 | Modified starch | 90.5% | 9.5% |
| 3 | Polyvinylpyrrolidone | 90.9% | 9.1% |
| | (“PVP”) | | |

### Composition for Stomach Release

The pharmaceutical compositions containing psilocybin can be suitable for stomach release so that the orally administered psilocybin can be absorbed by in the gastrointestinal tract.

In an embodiment, a pharmaceutical composition for stomach release may comprise psilocybin and chitosan, wherein the ratio of psilocybin to chitosan in a range of 1:10 to 1:11 of the pharmaceutical composition. In a further embodiment, the amount of psilocybin is about 9.6% w/w of the pharmaceutical composition.

Examples of pre-formulation compositions containing psilocybin as the API, which are suitable for stomach release are provided in Table 4 below.

TABLE 4
Pre-formulation compositions with various excipients for stomach release

| | | Excip. Conc. | Active conc. |
|---|---|---|---|
| Sample | Excipient | Wt. % | Wt. % |
| 1 | Chitosan | 90.4% | 9.6% |
| 2 | Eudragit E100 | 90.6% | 9.4% |
| 3 | Methyl cellulose | 89.1% | 10.9% |

### Methods of Manufacture

In certain embodiments, the pharmaceutical compositions containing psilocybin can be manufactured using the following exemplary processes.

### a) Chitosan/Eudragit E100 Solution:

An approx. 1-2 wt % excipient solution can be made using 1% acetic acid for dissolving the excipient. When the resulting solution is clear, the active ingredient can be added to attain a 10 wt % loading in the total formulation (calculated on solid bases).

### b) Methyl Cellulose/HPMC/Modified Starch/PVP:

An approx. 1-2 wt % excipient solution can be made using deionized water for dissolving the excipient. When the solution is clear, the active ingredient can be added to attain a 10 wt % loading in the total formulation (calculated on solid bases).

### c) Sc-CO₂c) Spray Drying Process and Process Conditions:

The solution obtained from Steps (a) or (b) above, as applicable, can be introduced to a pressure vessel filled with supercritical carbon dioxide (sc-CO₂) through a nozzle. The dried powder can be collected at the bottom of the pressure vessel or in collection chamber. The CO₂ in the pressure vessel can be maintained at 150 bar and 40° C. The CO₂ flow can be maintained between 680-720 l/h. The solution flow can be varied between 0.12-0.3 l/h.

The resulting pharmaceutical compositions containing psilocybin can be used as pre-formulations in the preparation of finished dosage forms suitable for oral administration, or more particularly, sublingual administration. As discussed in further detail below, the pharmaceutical compositions prepared in accordance with the disclosed manufacturing processes have a more uniform particle size as compared to unformulated psilocybin. Advantageously, pharmaceutical compositions containing comprising psilocybin can be provided in particulate form, wherein at least the formulated psilocybin is in particulate form. In embodiments, wherein the pharmaceutical compositions comprise further excipients in addition to those used to prepare the formulated psilocybin, the pharmaceutical compositions can be entirely in particulate form.

### Finished Dosage Forms

Any of the pharmaceutical compositions containing psilocybin disclosed herein can be used to provide finished dosage forms, including oral dosage forms (such as but not limited to tablets and capsules) and sublingual dosage forms, comprising said pharmaceutical compositions and optionally, one or more pharmaceutically acceptable excipients—including diluents, and/or carriers. The one or more pharmaceutically acceptable excipients (e.g. diluents and/or carriers) can be a solid component of the pharmaceutical dosage form.

Preferred compositions containing psilocybin comprise, based on a total of 100% by weight: (a) from about 10 to about 70% (preferably about 10%) of psilocybin; (b) from about 1 to about 70% diluent; (c) from about 2 to about 20% binder; (d) from about 1 to about 10% disintegrant; (e) from about 0.1 to about 5% anti-adherent; and (f) from about 0.2 to about 5% lubricant, and optionally (g) from about 0.2 to about 6% surfactant, and/or (h) up to about 2% (preferably, from about 0.1 to about 1%) colouring agent.

It will be appreciated that the finished dosage forms can be formulated to including varying dosages of the psilocybin. Preferably, the oral dosage form, or more particularly, the sublingual dosage form, contain about 25 mg to about 30 mg of psilocybin.

### Dissolution Performance of the Pharmaceutical Compositions

The pharmaceutical compositions containing psilocybin prepared in accordance with the disclosed manufacturing processes provide rapid dissolution in the expected time for the content to either be absorbed in the mucosal wall or pass to the stomach for absorption in the gastrointestinal tract.

In a particular embodiment, the pharmaceutical compositions are rapid release pharmaceutical composition, which are especially suitable for manufacturing tablets or capsules for oral administration. In an embodiment, the pharmaceutical composition comprises from about 10% by weight psilocybin (preferably about 5% to about 15% by weight psilocybin), and one or more pharmaceutically acceptable excipients (preferably chitosan, more preferably about 85% to about 95% by weight chitosan) and the pharmaceutical composition has a dissolution performance such that about 80% or greater, preferably 85% or greater of the psilocybin is released in the stomach within the first 5 minutes of oral administration.

In further embodiments, the pharmaceutical compositions are rapid release pharmaceutical composition, which are especially suitable for manufacturing sublingual dosage forms. In an embodiment, the pharmaceutical composition comprises from about 10% by weight psilocybin, and one or more pharmaceutically acceptable excipients (preferably polyvinylpyrrolidone, more preferably about 85% to about 95% by weight polyvinylpyrrolidone) and the pharmaceutical composition has a dissolution performance such that about 100% of the psilocybin is released in the mucosal wall within the first 1 minute of oral administration, preferably about 60-85% of the psilocybin is released in the mucosal wall within the first 30 seconds of oral administration, and more preferably, about 85% or greater of the psilocybin is released in the mucosal wall within the first 30 seconds of oral administration.

Quantification of Drug: UV-VIS analysis was chosen for the quantification of psilocybin in two media hydrochloric acid and phosphate buffer. UV-VIS was selected because it has the advantage of providing a rapid way to examine the API. Measurements were performed with a Thermo Scientific Helios Omega ultraviolet-visible spectroscope. Literature indicates two specific wavelengths for Psilocybin: 220-221 nm and 269 nm (Samuelsson et al, 2021, Anasto et al, 2005). For the purpose of analyzing the disclosed compositions, the reference the maximum absorption was selected at 269 nm. The calibration and all measurements were done at this specific wavelength.

The concentration of drug released was quantified for chitosan and polyvinylpyrrolidone samples in simulated gastric acid and simulated saliva, respectively. Samples were taken at five and four different times within 1 and 60 minutes (1, 5, 10 and 15 minutes and 5, 10, 15, 30 and 60 minutes) respectively. The analytical method described in the paragraph above was also used to measure the concentration of drug released as described herein. All tests were performed in triplicates.

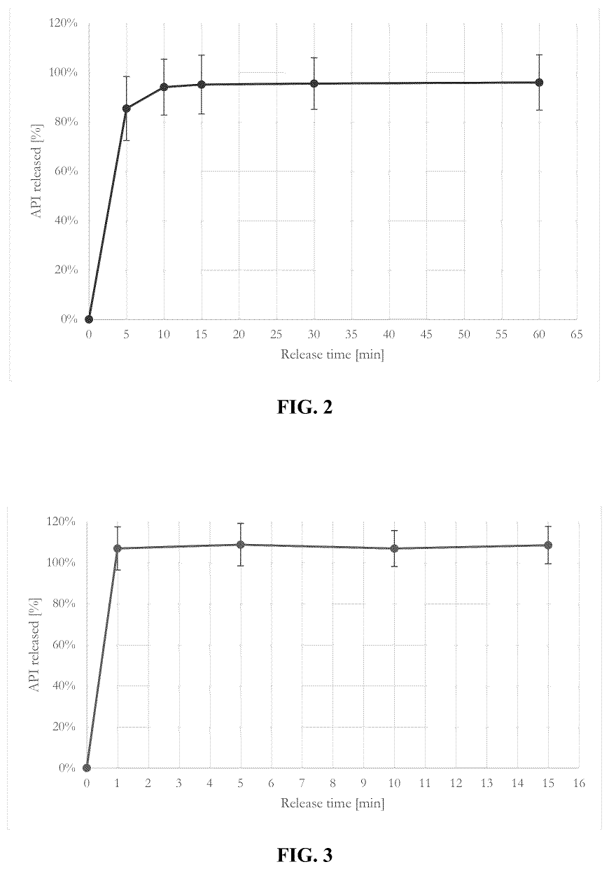

The release profiles show a quick release from the coating materials. As seen in FIG. 2, in the case of the chitosan-psilocybin composition more than 80% of the active ingredient is released within 5 minutes and almost 100% within 10 minutes.

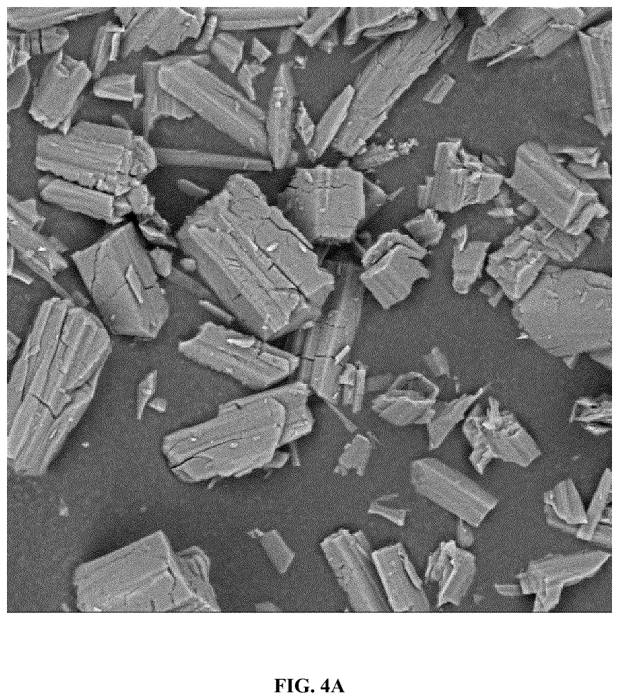

As seen in FIG. 3, in the case of PVP-psilocybin composition, the active ingredient is completely released within one minute.

The “dissolution performance” of the composition, as used herein with respect to psilocybin, refers to the weight % of psilocybin, based on the total weight of psilocybin contained in the pre-formulation, which is released within the defined period of time after oral administration under the following conditions: a known amount of sample pre-formulated powder (between 1-1.5 mg) was added to 10 ml sized bottle and 5 ml of simulated gastric acid or saliva for a total solution volume of 5 ml. The sample was stirred for 5 seconds with a Vortex mixer for each sample. Sampling was done at specified points of time as follows: (a) for the chitosan-psilocybin pre-formulation: 5, 10, 15, 30, and 60 minutes and (b) for PVP-psilocybin pre-formulation: 1, 5, 10 and 15 minutes. After the completion of the UV-VIS measurement the specimen was poured back to the bottle to maintain the volume in the bottle constant.

### Formulated Psilocybin Having Improved Uniform Particle Size

Further, unformulated psilocybin in its raw state, is highly irregular in shape without uniformity of particle size.

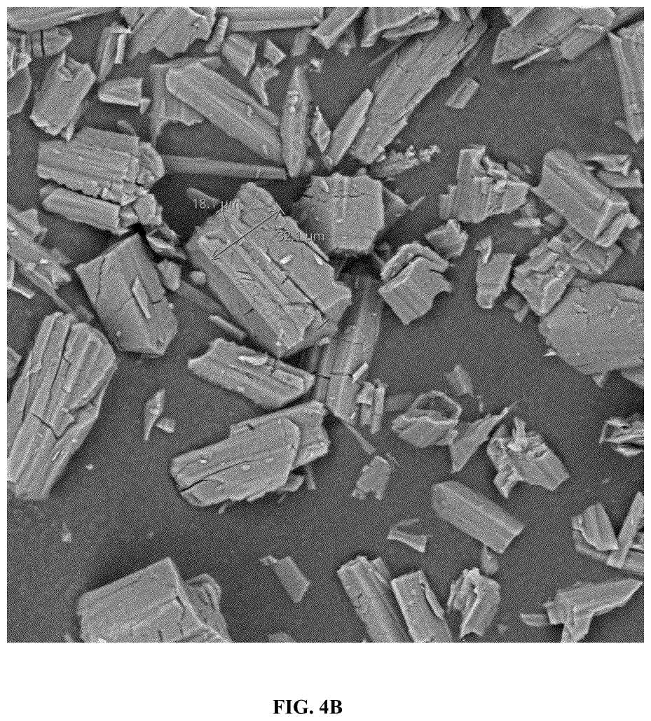

Scanning Electron Microscope micrographs of unformulated psilocybin in its raw state, depicted in FIG. 4A and FIG. 4B, were taken using a Thermo Fisher Scientific, Phenom ProX scanning electron microscope at a magnification about 2000-2100×.

The irregular shapes and sizes of psilocybin in its raw state means that pharmaceutical compositions containing unformulated psilocybin as the active compound can have widely varying drug release properties.

Further, the particle size of an active compound has been found to enhance the bioavailability and handling of pharmaceutical compositions containing psilocybin as described above. Accordingly, in a further aspect, disclosed are compositions comprising psilocybin in combination with one or more pharmaceutically acceptable excipients (such compositions are also referenced herein as “formulated psilocybin”), wherein the composition is processed by carbon dioxide spray drying. By employing carbon dioxide spray drying to dry the composition to fine particles, the resulting formulated psilocybin particles easily and rapidly dissolve in the mucosal wall or stomach but are stable in the dry state.

In a preferred embodiment, carbon dioxide spray drying can be performed in a 10-litre drying setup and take less than an hour to be completed. CO₂ and the solution containing the psilocybin and the one or more pharmaceutically acceptable excipients can be contacted at 150 bar and 40° C. in a procedure where all CO₂ is being recycled during the process. No heating of the solution is involved in the process and since oxygen is absent, even labile ingredients are preserved, without degradation or denaturation (in case of proteins). The small particle size, as determined above, can assist in a rapid dissolution and swift uptake when administered orally or sublingually.

Particle size analysis of the composition containing psilocybin and one or more pharmaceutically acceptable excipients (i.e. the formulated psilocybin) was conducted with Phenom Particle Metric software based on the SEM micrographs made with Phenom ProX. To compare the particles, the circle equivalent diameter for each particle, which means the diameter of a circle with the same surface area as the particle, was measured. This property is assessed based on particle count, which is the number of particles detected on the micrographs.

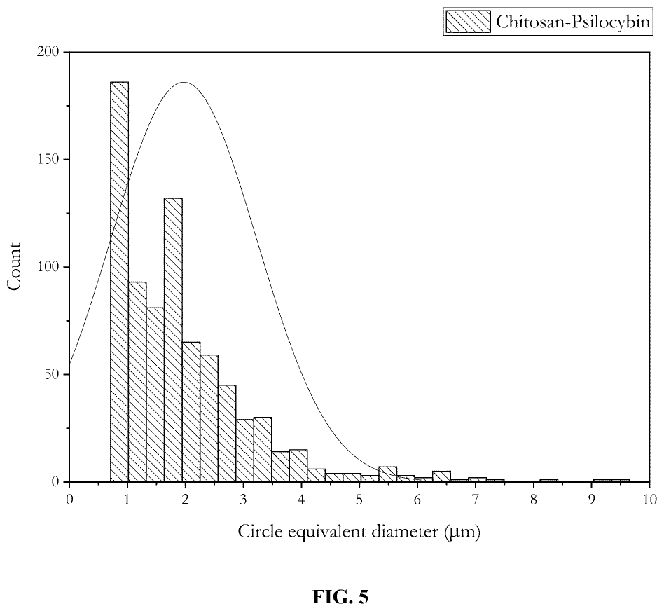

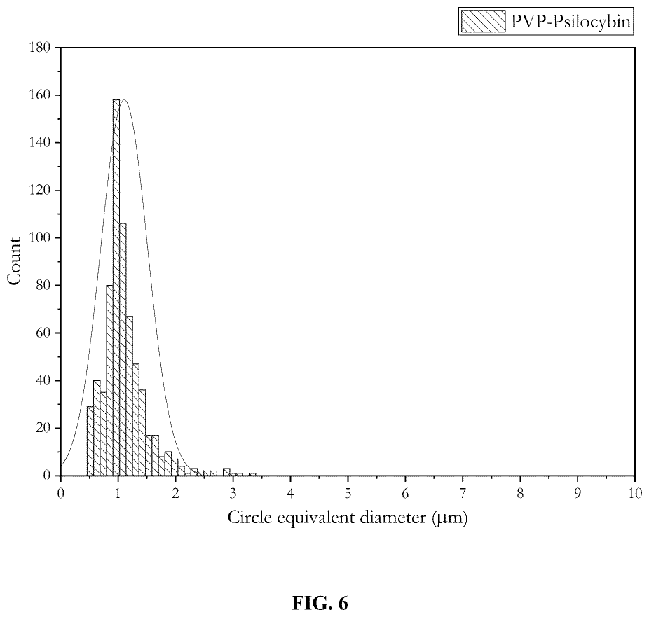

The particle size properties (size distribution, median and average) of compositions containing psilocybin in combination with chitosan or polyvinylpyrrolidone are reported Table 5 below and shown in FIG. 5 and FIG. 6. Chitosan creates a wider distribution and average size of particles, whereas polyvinylpyrrolidone shows the opposite. The average size of particles of polyvinylpyrrolidone is slightly more than half compared to the chitosan particles.

TABLE 5
Particle size measurements of the samples

| | | Mean circle equivalent diameter [mm] | |
|---|---|---|---|
| Sample name | Particle count | Median | Average |
| Chitosan-Psilocybin | 791 | 1.69 μm | 1.97 μm |
| PVP-Psilocybin | 680 | 1.02 μm | 1.10 μm |

As compared to the unformulated psilocybin in its raw state, which comprise uneven and large particles, the formulated psilocybin prepared in accordance with the process described above, displays a very narrow particle size distribution with a particle size average of 1.9 μm and 1.1 μm for chitosan coated psilocybin and polyvinylpyrrolidone coated psilocybin, respectively. The obtained compositions of formulated psilocybin are easy to handle (with free-flowing property), reproducible, and confirmed the expected loadings for use as pre-formulation compositions in the manufacture of tablets or capsules.

It has been surprisingly found that by processing psilocybin in combination with one or more excipients to bring the particle size within a particular narrow range, manufacturing capability is enhanced, and the resulting particles can be used to prepare pharmaceutical compositions that exhibit an improved bioavailability of the active ingredient psilocybin.

The defined particle size permits a uniform composition for stable pharmaceutical compositions. In a further aspect, disclosed are pharmaceutical compositions containing the formulated psilocybin, the pharmaceutical compositions exhibiting a rapid achievement of maximum blood concentration of psilocybin and/or a rapid onset of a therapeutic effect. Moreover, such pharmaceutical compositions, and in particular, compositions for use in the preparation of dosage forms for oral and sublingual administration, can be manufactured with proven reproducibility.

The nomenclature describing the particle size of the formulated psilocybin may be referred to as the “D90” or “d90” For example, a D90 of 2 (or d90=2) means that at least 90% of the particles have a particle size of less than 2 μm (microns).

In a further aspect, disclosed are particles of a composition comprising psilocybin and one or more pharmaceutically acceptable excipients, wherein at least 90% (D90) of the composition have a particle size of less than about 3 preferably between 1 to 3 and most preferably between 0.7-1.5

In one particular embodiment, the formulated psilocybin particles are particles of a chitosan-psilocybin composition have a D90 of about 3.37 μm and a D10 of about 811 nm, as measured by Scanning Electron Microscopy (“SEM”). The particles of the PVP-psilocybin composition have a D90 of about 1.54 μm and D10 of about 667 nm, as measured by SEM.

It is understood by those familiar with comminution process techniques that the limit set on the size of 90% or more of the particles, is a feature to further distinguish the disclosed particulate compounds from particles exhibiting a broader size distribution. Because of the variation in size encountered in all matter reduced in size by a comminution process, expressing differences in particle size in the manner described herein is readily accepted by those skilled in the art.

In an embodiment, particles of a composition comprising psilocybin can be used in the preparation of rapid release pharmaceutical compositions, which are especially suitable for manufacturing tablets or capsules for oral administration. In an embodiment, the rapid release pharmaceutical composition comprises from about 10% w/w of psilocybin, about 90% w/w of chitosan, and pharmaceutically acceptable excipients, said composition has a dissolution performance such that about 80% or greater, preferably 85% or greater of the psilocybin in said tablet or capsule is released in the stomach within the first 5 minutes of oral administration, wherein at least 90% (D90) the pharmaceutical composition containing psilocybin have a particle size of less than 3.37 μm, preferably at least 90% (D90) the pharmaceutical composition containing psilocybin have a particle size of between 1 to 3 μm, and more preferably at least 90% (D90) the pharmaceutical composition containing psilocybin have a particle size of between 0.7-1.5 μm.

In a further embodiment, particles of a composition comprising psilocybin can be used in the preparation of rapid release pharmaceutical compositions, which are especially suitable for manufacturing a dosage form for sublingual administration. In an embodiment, the rapid release pharmaceutical composition comprises from about 10% by weight psilocybin, about 90% of polyvinylpyrrolidone, and pharmaceutically acceptable excipients, said composition has a dissolution performance such that about 100% of the psilocybin is released in the mucosal wall within the first 1 minute of oral administration, wherein at least 90% (D90) the pharmaceutical composition containing psilocybin have a particle size of less than 1.54 μm, and preferably at least 90% (D90) the pharmaceutical composition containing psilocybin have a particle size of between 0.7-1.5 μm.

### EXAMPLES

The following tables recite preferred compositions containing psilocybin, which can be used to produce tablets or capsules of especially high quality and superior performance.

Table 6 recites preferred compositions containing psilocybin for rapid mucosal release.

TABLE 6
Composition of PVP-psilocybin for mucosal release

| Excipient | Excip. conc. wt. % | Active conc. Wt. % |
|---|---|---|
| PVP | 90.9% | 9.1% |

Table 7 recites preferred compositions containing psilocybin for rapid stomach release.

TABLE 7
Composition of Chitosan-psilocybin for stomach release

| Excipient | Excip. conc. wt. % | Active conc. Wt. % |
|---|---|---|
| Chitosan | 90.4% | 9.6% |

These Examples are provided to illustrate preferred embodiments and are not intended to limit the scope of the present claims.

### REFERENCES

Brown, R. T., Nicholas, C. R., Cozzi, N. V., Gassman, M. C., Cooper, K. M., Muller, D., Hutson, P. R. (2017). Pharmacokinetics of Escalating Doses of Oral Psilocybin in Healthy Adults. Clin Pharmacokinet. doi:10.1007/s40262-017-0540-6.
Carhart-Harris, R. L., Leech, R., Hellyer, P. J., Shanahan, M., Feilding, A., Tagliazucchi, E., Nutt, D. (2014). The entropic brain: a theory of conscious states informed by neuroimaging research with psychedelic drugs. Front Hum Neurosci, 8, 20. doi:10.3389/fnhum.2014.00020
Chen, J., Li, M., Yan, X., Wu, E., Zhu, H., Lee, K. W., Chu, V. M., et al. (2011). Determining the pharmocokinetics of psilocin in rat plasma using ultra-performance liquid chromatography coupled with a photodiode array detector after orally administering an extract of Gymnopilus spectabilis. J Chromatogr B. 879(2011), 2669-2672.
Geyer, M. A., & Vollenweider, F. X. (2008). Serotonin research: contributions to understanding psychoses. Trends Pharmacol Sci, 29(9), 445-453. doi :10.1016/j .tips.2008.06.006
Griffiths, R. R., Johnson, M. W., Carducci, M. A., Umbricht, A., Richards, W. A., Richards, B. D., Klinedinst, M. A. (2016). Psilocybin produces substantial and sustained decreases in depression and anxiety in patients with life-threatening cancer: A randomized double-blind trial. J Psychopharmacol, 30(12), 1181-1197. doi:10.1177/0269881116675513
Hasler, F., Bourquin, D., Brenneisen, R., Bar, T., & Vollenweider, F. X. (1997). Determination of psilocin and 4-hydroxyindole-3-acetic acid in plasma by HPLC-ECD and pharmacokinetic profiles of oral and intravenous psilocybin in man. Pharm Acta Hely, 72(3), 175-184.
Hasler, F., Bourquin, D., Brenneisen, R., & Vollenweider, F. X. (2002). Renal excretion profiles of psilocin following oral administration of psilocybin: a controlled study in man. J Pharm Biomed Anal, 30(2), 331-339.
Hopf, A., & Eckert, H. (1969). Autoradiographic studies on the distribution of psychoactive drugs in the rat brain. 3. 14C-psilocin. Psychopharmacologia, 16(3), 201-222.
Horita, A., & Weber, L. J. (1962). Dephosphorylation of psilocybin in the intact mouse. Toxicology and Applied Pharmacology, 4(6), 730-737. https://doi.org/10.1016/0041-008X(62)90102-3
Kalberer, F., Kreis, W., Rutschmann, J. (1962). The fate of psilocin in the rat. Biochemical Pharmacology. 11, 261-269.
MacLean, K. A., Johnson, M. W., & Griffiths, R. R. (2011). Mystical experiences occasioned by the hallucinogen psilocybin lead to increases in the personality domain of openness. J Psychopharmacol, 25(11), 1453-1461. doi:10.1177/0269881111420188
Madsen, M. K., Fisher P.M., Burmester D., Dyssegaard A., Stenbaek, D. S. Kristiansen S., Johansen, S. S., Lehel, S. Linnet, K., Svarer, C., Erritzoe, D., Ozenne, B., & Knudsen G. M. Psychedelic effects of psilocybin correlate with serotonin 2A receptor occupancy and plasma psilocin levels (2019). Neuropsychopharmacol, 44, 1328-1334. https://doi.org/10.1038/s41386-019-0324-9
Manevski, N., Kurkela, M., Hoglund, C., Mauriala, T., Court, M. H., Yli-Kauhaluoma, J., & Finel, M. (2010). Glucuronidation of psilocin and 4-hydroxyindole by the human UDP-glucuronosyltransferases. Drug Metab Dispos, 38(3), 386-395. doi:10.1124/dmd.109.031138
N. Anastos, S. W. Lewis, N. W. Barnett, and D. Sims, “The determination of psilocin and psilocybin in hallucinogenic mushrooms by HPLC utilizing a dual reagent acidic potassium permanganate and tris(2,2′-bipyridyl)ruthenium(II) chemiluminescence detection system,” Journal of Forensic Sciences, vol. 51, no. 1, pp. 45-51, January 2006, doi:10.1111/j.1556-4029.2005.00033.x.
Nichols, D. E. (2004). Hallucinogens. Pharmacology & Therapeutics, 101(2), 131-181. doi:10.1016/j.pharmthera.2003.11.002
Passie, T., Seifert, J., Schneider, U., & Emrich, H. M. (2002). The pharmacology of psilocybin. Addict Biol, 7(4), 357-364. doi :10.1080/1355621021000005937
Ross, S., Bossis, A., Guss, J., Agin-Liebes, G., Malone, T., Cohen, B., Schmidt, B. L. (2016). Rapid and sustained symptom reduction following psilocybin treatment for anxiety and depression in patients with life-threatening cancer: a randomized controlled trial. J Psychopharmacol, 30(12), 1165-1180. doi:10.1177/0269881116675512
Samuelsson A, Janusson E, Shah S, Roggen M. Rapid quantification of Psilocybin with reversed-phase HPLC and single-wavelength detection. ChemRxiv. Cambridge: Cambridge Open Engage; 2021. https://doi.org/10.26434/chemrxiv-2021-70 mm1
Studerus, E., Kometer, M., Hasler, F., & Vollenweider, F. X. (2011). Acute, subacute and long- term subjective effects of psilocybin in healthy humans: a pooled analysis of experimental studies. J Psychopharmacol, 25(11), 1434-1452. doi:10.1177/0269881110382466

What is claimed is:

1. A pharmaceutical composition comprising,
(a) psilocybin in an amount of about 5% w/w to about 15% w/w of the pharmaceutical composition; and
(b) chitosan in an amount of about 85% w/w to about 95% w/w of the pharmaceutical composition;
wherein the pharmaceutical composition has a dissolution performance such that about 80% or greater of the psilocybin in the pharmaceutical composition is released in a subject's stomach within the first 5 minutes of oral administration.

2. The pharmaceutical composition according to claim 1, comprising, (a) psilocybin in an amount of about 9.4% w/w to about 10.9% w/w of the pharmaceutical composition; and
(b) chitosan in an amount of about 89.1% w/w to about 90.6% w/w of the pharmaceutical composition.

3. The pharmaceutical composition of claim 1, wherein the pharmaceutical composition has a dissolution performance such that about 85% or greater of the psilocybin in said pharmaceutical composition is released in a subject's stomach within the first 5 minutes of oral administration.

4. The pharmaceutical composition of claim 1, wherein the pharmaceutical composition is in particulate form, and wherein at least 90% (D90) of the pharmaceutical composition has a particle size of less than 3.37 μm.

5. The pharmaceutical composition of claim 1, wherein the pharmaceutical composition is in particulate form, and wherein at least 90% (D90) of the pharmaceutical composition has a particle size of less than 1.54 μm.

6. The pharmaceutical composition according to claim 1, wherein pharmaceutical composition further comprises one or more pharmaceutically acceptable excipients.

7. The pharmaceutical composition according to claim 6, wherein the one or more pharmaceutically acceptable excipients are selected from the group consisting of:
binders, disintegrants, lubricants, flowing agents, glidants, anti-adherents, surfactants, stabilizers, binders, bulking agents, colouring agents, and coating agents.

8. The pharmaceutical composition according to claim 1, wherein the pharmaceutical composition is in a unit dosage form that is an oral dosage form.

9. The pharmaceutical composition of claim 8, wherein the oral dosage form contains about of about 25 to about 30 mg psilocybin.

10. The pharmaceutical composition of claim 8, wherein the oral dosage form is a tablet or a capsule.

11. A pharmaceutical composition comprising,
(a) psilocybin in an amount of about 5 to about 15% w/w of the pharmaceutical composition; and
(b) polyvinylpyrrolidone in an amount of about 85 to about 95% w/w of the pharmaceutical composition;
wherein the pharmaceutical composition has a dissolution performance such that about 100% of the psilocybin in the pharmaceutical composition is released in a subject's oral mucosal wall within the first 1 minute of oral administration.

12. The pharmaceutical composition according to claim 11, comprising,
(a) psilocybin in an amount of 9.1% w/w of the pharmaceutical composition; and
(b) polyvinylpyrrolidone in an amount of 90.9% w/w of the pharmaceutical composition.

13. The pharmaceutical composition of claim 11, wherein the pharmaceutical composition has a dissolution performance such that about 85% or greater of the psilocybin in the pharmaceutical composition is released in a subject's oral mucosal wall within the first 30 seconds of oral administration.

14. The pharmaceutical composition of claim 11, wherein the pharmaceutical composition is in particulate form, and wherein at least 90% (D90) of the pharmaceutical composition has a particle size of less than 1.54 μm.

15. The pharmaceutical composition of claim 11, wherein the pharmaceutical composition is in particulate form, and wherein at least 90% (D90) of the pharmaceutical composition has a particle size between 0.7-1.5 μm.

16. The pharmaceutical composition according to claim 11, wherein pharmaceutical composition further comprises one or more pharmaceutically acceptable excipients.

17. The pharmaceutical composition according to claim 16, wherein the one or more pharmaceutically acceptable excipients are selected from the group consisting of: binders, disintegrants, lubricants, flowing agents, glidants, anti-adherents, surfactants, stabilizers, binders, bulking agents, colouring agents, and coating agents.

18. The pharmaceutical composition according to claim 11, wherein the pharmaceutical composition is in a unit dosage form that is a sublingual dosage form.

19. The pharmaceutical composition according to claim 18, wherein the sublingual dosage form contains about 25 to about 30 mg of the psilocybin.

20. The pharmaceutical composition according to claim 18, wherein sublingual dosage form is a tablet, a wafer, or a film.
